(19) United States
(12) Patent Application Publication
(10) Pub. No.: US 2005/0119659 A1
(43) Pub. Date: Jun. 2, 2005

(54) Distractor

(76) Inventors: Joachim Pfefferle, Munstertal (DE); Dominik Burkhart, Lausen (CH); Peter Scheuble, Schliengen (DE); Andreas Stricker, Konstant (DE)

Correspondence Address: WELSH & KATZ, LTD, 120 S RIVERSIDE PLAZA, 22ND FLOOR, CHICAGO, IL, 60606, US

(21) Appl. No.: 10/510,366

(22) PCT Filed: Mar. 28, 2003
(86) PCT No.: PCT/CH03/00201

(30) Foreign Application Priority Data
Apr. 9, 2002 (CH) 600/02

(51) Int. Cl. A61B 17/56

(52) U.S. Cl. 606/71

(57) ABSTRACT

A distractor for osteogenesis, in particular in the front region of the lower or upper jaw, with first fixation means for fixation to a first, fixed bone fragment, and second fixation means for fixation to a second bone fragment, which is movable in relation to the first bone fragment in a defined plane, and also having an adjustment element connected to the first fixation means and to the second fixation means, and by the actuation of which the distance between the first fixation means and the second fixation means is variable, whereby the bone fragments to which the first fixation means and the second fixation means can be fixed can be moved away from or toward each other, wherein the first fixation means and the adjustment element are formed or connected to each other such that when the adjustment element is actuated, the second fixation means, which can be fixed to the movable bone fragment, can be pivoted out of the plane in which they can be fixed to the bone fragment.

### FIELD OF INVENTION

The invention relates to a distractor for use with correcting trauma to the mouth, face and jaw.

### BACKGROUND

Distractors are used in osteosynthesis/osteogenesis, where they are used in particular in the region of the mouth, jaw and face. A special field of application of such distractors lies in the front region of the jaw, for example the lower jaw (mandibula), or else in the front region of the upper jaw (maxilla). If, for example, the lower jawbone is atrophic in the front region, or if a segment of the lower jaw had to be cut away on account of malignant neoplasms, or else after a lower jaw trauma, it is favorable for a reconstruction of the jaw with the aid of dental implants or prostheses if the alveolar ridge of the lower jaw in the front region can first be reconstructed before the implants are set.

This typically takes place by the upper bone segment being mobilized (osteotomy, corticotomy) in the front region of the lower jaw—where the bone defect is to be rectified. With the aid of a distractor, the mobilized bone segment is then raised slightly from the fixed lower jawbone. The osteoinductive effect of the distraction causes new bone to be formed between the raised bone segment and the fixed bone. The raising of the movable bone segment is in this case performed in small increments, for as long as it takes for the alveolar ridge to be reconstructed. The gingiva (gum tissue) covering the osseous alveolar ridge is likewise raised at the same time by the distraction.

Previously known distractors used in such applications typically comprise two bone plates (typically so-called miniplates), one of which is fixed (for example by screwing) to the mobilized (by osteotomy, corticotomy) bone segment and the other of which is fixed to the fixed lower jawbone. An adjustment element (distraction spindle) is connected to both bone plates. With the aid of the adjustment element, the distance between the two bone plates can be varied. With appropriate actuation of the adjustment element, that bone plate which is fixed to the mobilized bone segment is moved linearly along the spindle axis away from that bone plate which is fixed to the fixed lower jawbone.

This tried-and-tested distraction procedure is in need of improvement, however, to the extent that the distraction only takes place strictly in the direction of the spindle axis. Since, however, the lower jaw has a certain natural curvature—referring here not to the curvature of the lower jaw in the horizontal plane but to the curvature of the lower jaw (when viewed from the front) in the sagittal planes—this curvature of the lower jaw cannot be optimally replicated by the known distractors, because of the fact that the distraction can only take place strictly in the direction of the spindle axis. Furthermore, the tensile force of the labial frenulum also acts in the front region, so that it is especially difficult there for the alveolar ridge to be reconstructed as faithfully as possible. However, it is specifically in the front region of the lower jaw (and also in the front region of the upper jaw) that the most faithful possible reconstruction of the alveolar ridge is particularly desired.

### SUMMARY

This is where the present invention comes in, the object of which is to propose a distractor with which it is intended to be possible in osteogenesis by means of distraction to permit the most optimum possible shaping of the bone to be built up, and in particular in the reconstruction of an alveolar ridge of the lower or upper jaw to permit the replication of the natural curvature of the jaw. The distractor is preferably to be made in a form simply to produce.

This object is achieved by the distractor according to the invention as it is characterized by the features of the independent patent claim. Particularly advantageous refinements of the distractor according to the invention emerge from the features of the dependent patent claims.

In particular, the distractor comprises first fixation means, for fixation to a first, fixed bone fragment, and second fixation means, for fixation to a second bone fragment, which is movable in relation to the first bone fragment, in a defined plane. The distractor also comprises an adjustment element, which is connected to the first fixation means on the one hand and to the second fixation means on the other hand, and by the actuation of which the distance between the first fixation means and the second fixation means is variable. This allows the bone fragments to which the first and second fixation means can be fixed to be moved away from or toward each other. The first fixation means and the adjustment element are formed or connected to each other in such a way that, when the adjustment element is actuated, the second fixation means, which can be fixed to the movable bone fragment, can be pivoted out of the plane in which they can be fixed to the bone fragment.

This pivotability of the second fixation means out of the plane in which they are fixed to the movable bone fragment allows the natural construction of the alveolar ridge, for example of the lower jaw, because the direction in which the movable bone fragment (mobilized by osteotomy or corticotomy) is distracted in relation to the fixed (immobile) bone fragment can be changed during the buildup of the bone (osteogenesis) by distraction. This allows the natural curvature of the jaw to be taken into account and to be replicated as faithfully as possible in the osteogenesis.

In the case of an exemplary embodiment of the distractor according to the invention, the first fixation means comprise a first bone plate and the second fixation means comprise a second bone plate, the adjustment element being connected to the first bone plate on the one hand and to the second bone plate on the other hand. Fixation with the aid of bone plates (in particular with so-called miniplates) is a particularly reliable method of fixation, which can also be mastered very well with respect to the surgical procedures involved. However, fixation by other suitable means (for example pins, etc.) could also be considered in principle, although fixation by means of bone plates is preferred.

The first bone plate may in this case be formed in such a way that it has a longitudinal plate axis, the second bone plate being able to pivot about this longitudinal plate axis or about an axis parallel to it. As already mentioned above—this pivotability of the second bone plate about the longitudinal plate axis of the first bone plate or about an axis parallel to it allows the natural construction of the alveolar ridge, for example of the lower jaw, because the direction in which the movable (mobilized) bone fragment is distracted in relation to the fixed (immobile) bone fragment can be changed during the buildup of the bone (osteogenesis) by distraction. This allows the natural curvature of the jaw to be replicated as faithfully as possible in the osteogenesis.

In the case of an advantageous exemplary embodiment of the distractor according to the invention, the adjustment element is connected to the first fixation means or the first bone plate in the manner of a hinge. The hinge-like connection defines the pivot axis about which the second fixation means or the second bone plate are or is pivotable.

In the case of an advantageous development of the distractor according to the invention, the hinge is formed as a film hinge. This type of formation of the hinge is particularly simple in terms of production engineering.

In the case of a further advantageous development of the distractor according to the invention, a fixation element is provided which is connected via the hinge to the first fixation means or the first bone plate and which has via the adjustment element a rigid connection to the second fixation means or the second bone plate. The adjustment element comprises a pivoting element for pivoting the second fixation means or the second bone plate, which is connected to the fixation element on the one hand and to the first fixation means or the first bone plate on the other hand.

In this case, two or more thin webs, which form the hinge, may be provided between the fixation element and the first fixation means or the first bone plate. Two or more webs in this case prevent tilting of the fixation element and consequently tilting of the second fixation means or the second bone plate within their plane.

The fixation element and the hinge may be formed in one piece on the first fixation element or the first bone plate, which simplifies production to the extent that the bone plate, the fixation element, and in the case of webs, also the webs can be produced in one and the same production operation and do not have to be produced separately and then connected to one another in a suitable way.

In the case of a development of the distractor according to the invention, the adjustment element comprises a distraction element for varying the linear distance between the first and second fixation means or the first and second bone plates, and also the pivoting element for pivoting the second bone plate, a rigid connection existing between the distraction element and the pivoting element. A first retaining element is fixed to the fixation element and a second retaining element is fixed to the first fixation means or the first bone plate. The pivoting element has a threaded spindle and the first retaining element, which is fixed to the fixation element, has a through-bore with a thread for receiving the threaded spindle. The threaded spindle is received in such a way that it cannot be displaced in the direction of the spindle axis in the second retaining element, which is fixed to the first fixation means or to the first bone plate. This development represents a variant of the distractor according to the invention that is advantageous in terms of constructional design and favorable in terms of production. It is also advantageous to the extent that the “overall height” of the distractor is maintained, and does not increase, during pivoting.

In the case of a further exemplary embodiment of the distractor according to the invention, the hinge is formed in such a way that the second fixation means or the second bone plate is pivotable in relation to the first fixation means or to the first bone plate by an absolute angle of up to 30°, preferably of up to 15° (both pivoting directions). This range is large enough to replicate the natural curvatures in particular in the jaw region.

The distractor according to the invention is preferably produced from titanium or some other biocompatible material, in order that it does not cause any reactions of the body.

### BRIEF DESCRIPTION OF THE DRAWINGS

Further advantageous refinements of the distractor according to the invention emerge from the description of an advantageous exemplary embodiment which follows with the aid of the drawing, in which:

FIG. 1 shows an exemplary embodiment of a specific embodiment of a distractor according to the invention in a front view;

FIG. 2 shows the distractor from FIG. 1 in a rear view;

FIG. 3 shows the distractor from FIG. 1 in a perspective view from the rear;

FIG. 4 shows the distractor from FIG. 1 in a perspective view from the front (partly in section);

FIG. 5 shows the distractor from FIG. 1 in a side view in the neutral position (partly in section);

FIGS. 6-8 show the distractor from FIG. 1 in three different pivoting positions; and

FIGS. 9-11 show the distractor from FIG. 1 in three further pivoting positions.

### DETAILED DESCRIPTION

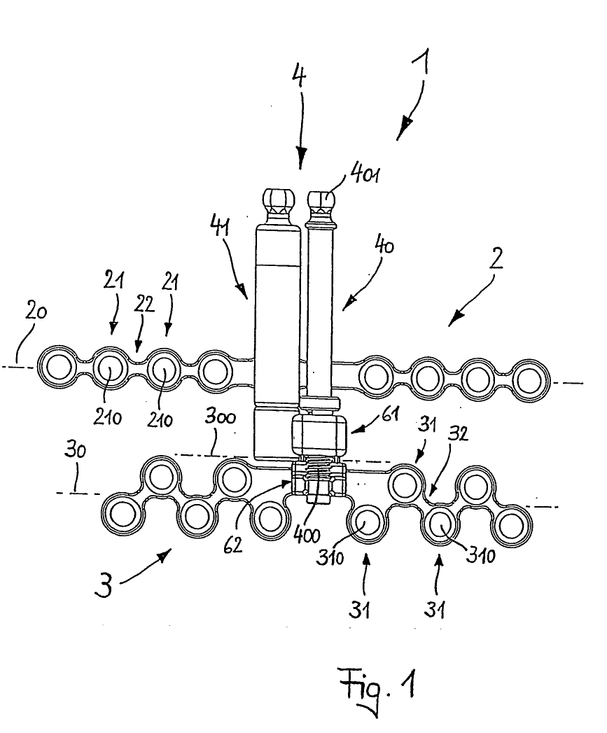

FIG. 1 shows a front view of an exemplary embodiment of a distractor 1 according to the invention, as can be used in particular in the front region of the lower or upper jaw. The distractor 1 comprises a first bone plate 3 and a second bone plate 2, which are in each case preferably formed as an elongate miniplate. The second bone plate 2 extends in the direction of its longitudinal plate axis 20. It has a number of eyelets 21, through which bone screws (not represented) can be screwed into the movable bone segment, in order to fix it to the movable bone segment. For this purpose, the plate holes 210 may be formed as through-holes; they may have a thread, which interacts with a corresponding screw thread, or they may have an engagement contour differing from a thread, for blocking the bone screw. Such an engagement contour and the way in which it functions are described for example in WO-A-00/66012. Provided between the eyelets 21 of the bone plate 2 are webs 22, which allow separation (cutting away) and/or bending of the bone plate 2 to the respective outer contour of the movable bone segment.

The first bone plate 3 is likewise formed in an elongated manner and extends in the direction of its longitudinal plate axis 30. In principle, the first bone plate 3 may also have the same shape as the second bone plate 2. The first bone plate 3 likewise has a number of eyelets 31, provided in which are plate holes 310, which may in turn be formed as through-holes, may have a thread for interaction with a corresponding screw thread, or which may have an engagement contour differing from a thread, as described for example in the aforementioned WO-Ab 00/66012. Webs 32 are provided between the eyelets 31, so that the first bone plate can also be separated (cut away) and/or bent to the jawbone well (to be precise to a fixed bone segment of the jawbone).

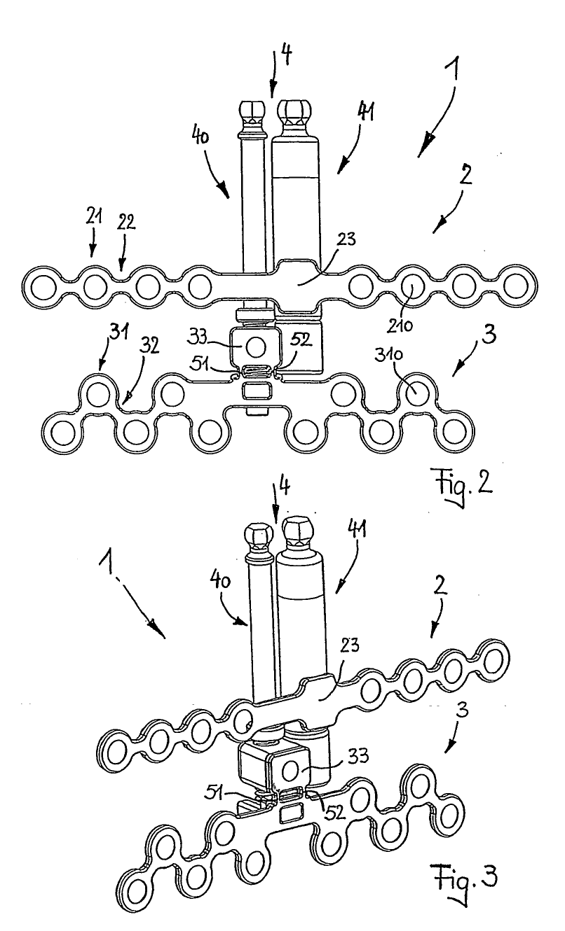

The distractor 1 also comprises an adjustment element 4, which for its part comprises a separate pivoting element 40 and a separate distraction element 41. The pivoting element 40 and the distraction element 41 are rigidly connected to each other. Provided on the second bone plate 2 is a platelet 23 (see for example FIG. 2, FIG. 3), to which the distraction element 41 (distraction spindle) is firmly connected, for example welded.

Provided on the first bone plate 3 is a fixation element, which here has the form of a platelet 33 (see for example FIG. 2, FIG. 3), which is connected to the first bone plate 3 in the manner of a hinge. In the case of the exemplary embodiment represented, the hinge is a film hinge, which here has two thin webs 51 and 52. Two such webs 51 and 52 are advantageous to the extent that this very greatly hinders, if not prevents, tilting of the second bone plate 2 in the plane of the plate (here the plane of the paper). More than two webs may also be provided for this purpose, but in principle a single web would also be sufficient to perform the hinge function (and could then be formed somewhat wider than in the case of the two-web variant represented). The hinge could also be formed differently than a film hinge, as long as the hinge function is ensured. With the aid of the hinge, here with the aid of the webs 51 and 52, the second bone plate 2 in the exemplary embodiment shown is pivotable about an axis 300 parallel to the longitudinal plate axis 30 of the first bone plate 3. That is to say that the second bone plate 2 in FIG. 1 can be pivoted out of the plane of the paper to the front or to the rear about the axis 300, as explained more precisely further below.

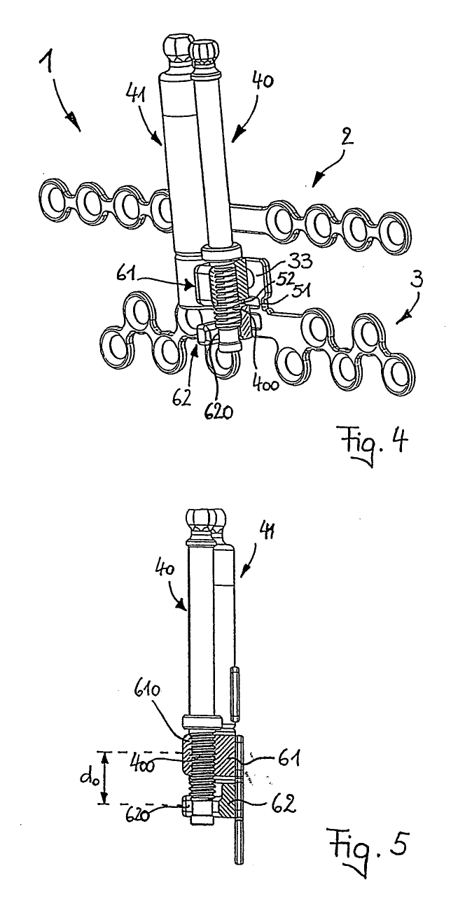

Provided on the fixation element—the platelet 33—is a first retaining element 61 and provided on the first bone plate 3 is a second retaining element 62, which can in each case be produced as separate parts and fixed to the platelet 33 or to the bone plate 3, for example by being welded on. Provided in the first retaining element 61 is a through-bore with a thread 610 (see FIG. 5), into which the thread of a threaded spindle 400 of the pivoting element 40 can engage. The second retaining element 62 has a clearance 620 (FIG. 4, FIG. 5), in which the distal end of the threaded spindle 400 is received in such a way that it cannot be displaced in the direction of the spindle axis (see FIG. 1). On the other hand, the distal end of the threaded spindle 400 can otherwise be displaced very well within certain limits in the clearance 620, substantially to the front or rear, as will become evident further below from the description of the way in which the distractor 1 functions.

The way in which the distractor 1 functions, in particular when pivoting, is to be explained below on the basis of FIGS. 6-8 and FIGS. 9-11. This explanation concentrates on the pivoting about the axis 300 (see FIG. 1), that is the change of direction, while the linear distraction—that is the change of the distance between the two bone plates—takes place in a known way by means of actuation of the distraction element 41 (distraction spindle).

To understand the way in which the distractor functions during the pivoting, let us consider once again FIG. 5, in which the distractor 1 is located in the neutral position (that is unpivoted). As a difference from this neutral position, the distractor in FIG. 6 is in a position in which the platelet 33 has been pivoted about an angle of, for example, α₁=5° in relation to its neutral position. This pivoting can be achieved by actuating the pivoting element 40, which may take place here with the aid of a suitable wrench (not represented), which acts on the polygonal head 401 of the threaded spindle 400, so that the threaded spindle 400 can be turned. When the polygonal,head 401 of the threaded spindle turns, the threaded spindle 400 attempts—by the interaction of the thread of the threaded spindle 400 with the thread 610 of the first retaining element 61—to increase the distance do between the first retaining element 61 and the second retaining element 62 in the axial direction. In the neutral position, however, this is not possible in the direction of extent of the spindle axis, because, as already described above, the distal end of the threaded spindle 400 is captured in the clearance 620 of the second retaining element 62 in such a way that it cannot be displaced in the axial direction. On the other hand, the turning of the threaded spindle 400 must cause the distance do which exists between the first retaining element 61 and the second retaining element 62 in the neutral position to be increased, otherwise the threaded spindle 400 would not allow itself to be turned with a captured distal end. Since, however, the webs 51 and 52 of the film hinge are flexible, the webs 51 and 52 are bent by the turning of the threaded spindle 400, and so the platelet 33 with the first retaining element 61 fixed to it is pivoted about the axis of rotation 300 (see FIG. 1). As a result, the distance d₀ between the two retaining elements 61 and 62 in the neutral position (FIG. 5) is increased to the distance d₁ in the position according to FIG. 6 (pivoting of the spindle axis by 50). As this takes place, the distal end of the threaded spindle slides slightly outward in the clearance 620 of the second retaining element 62.

With further turning of the threaded spindle 400, the platelet 33 with the first retaining element 61 fixed to it comes into the position shown in FIG. 7, in which the platelet 33 has been pivoted about the angle α₂=10° with respect to the neutral position about the axis 300 (FIG. 1). The distance between the first retaining element 61 and the second retaining element 62 in the direction of the spindle axis is thereby increased to the distance d₂.

If the threaded spindle 400 is turned further, the platelet 33 with the first retaining element 61 fixed to it finally comes into the position shown in FIG. 8, in which the platelet 33 has been pivoted by the angle α₃=15° with respect to the neutral position about the axis 300 (see FIG. 1). The distance between the first retaining element 61 and the second retaining element 62 in the direction of the spindle axis is thereby increased to the distance d₃.

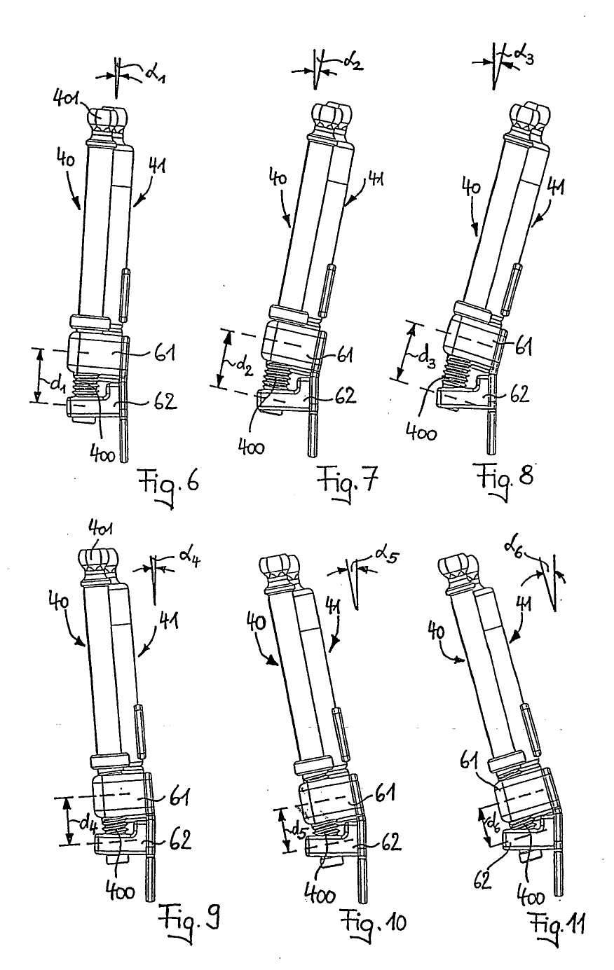

The pivoting of the platelet 33 in the other direction functions in-the same way, as FIGS. 9-11 show in a simple way, the distance do (neutral position according to FIG. 5) between the first retaining element 61 and the second retaining element 62 initially being reduced there to the distance d₄ with a pivoting angle α₄=5° of the platelet 33, which distance is reduced further to the distance d₅ with a pivoting angle of α₅=10°, and which distance is finally reduced to the distance d₆ with a pivoting angle of α₆=15°.

It is particularly advantageous in the case of the distractor 1 according to the invention that the individual parts, that is the bone plates 2 and 3 together with the platelet 33 and the webs 51 and 52, as well as the retaining elements 61 and 62, and also the pivoting element 40 and the distraction element 41, can be produced separately in a simple and reliable way and can be joined together, for example by means of welding, so that the distractor according to the invention can be mastered well in terms of production.

Titanium, which is known to be biocompatible, comes into consideration in particular as the material for the individual parts. Alternatively, other biocompatible materials may also be used, however, and bioresorbable materials may also be considered.

Specific embodiments of a Distractor according to the present invention have been described for the purpose of illustrating the manner in which the invention may be made and used. It should be understood that implementation of other variations and modifications of the invention and its various aspects will be apparent to those skilled in the art, and that the invention is not limited by the specific embodiments described. It is therefore contemplated to cover by the present invention any and all modifications, variations, or equivalents that fall within the true spirit and scope of the basic underlying principles disclosed and claimed herein.

1. A distractor for osteogenesis, in particular in the front region of the lower or upper jaw, with first fixation means for fixation to a first, fixed bone fragment, and second fixation means for fixation to a second bone fragment, which is movable in relation to the first bone fragment in a defined plane, and also having an adjustment element connected to the first fixation means and to the second fixation means, and by the actuation of which the distance between the first fixation means and the second fixation means is variable, whereby the bone fragments to which the first fixation means and the second fixation means can be fixed can be moved away from or toward each other, wherein the first fixation means and the adjustment element are formed or connected to each other such that when the adjustment element is actuated, the second fixation means, which can be fixed to the movable bone fragment, can be pivoted out of the plane in which they can be fixed to the bone fragment.

2. The distractor according to claim 1, wherein the first fixation means comprise a first bone plate and the second fixation means comprise a second bone plate, the adjustment element being connected to the first bone plate and to the second bone plate.

3. The distractor according to claim 2, wherein the first bone plate has a longitudinal plate axis and the second bone plate can pivot about the longitudinal plate axis or about an axis parallel to it.

4. The distractor according to claim 1 wherein the adjustment element is connected to the first fixation means or to the first bone plate in the manner of a hinge.

5. The distractor according to claim 4, wherein the hinge is formed as a film hinge.

6. The distractor according to claim 4, including a fixation element connected via the hinge to the first fixation means or to the first bone plate and which has via the adjustment element a rigid connection to the second fixation means or the second bone plate, and wherein the adjustment element comprises a pivoting element for pivoting the second fixation means or the second bone plate, which is connected to the fixation element and to the first fixation means or the first bone plate.

7. The distractor according to claim 6, further including two or more thin webs, which form the hinge, provided between the fixation element and the first fixation means or the first bone plate.

8. The distractor according to claim 6, wherein the fixation element and the hinge are formed in one piece on the first fixation means or the first bone plate.

9. The distractor according to claim 6, wherein the adjustment element comprises a distraction element for varying the linear distance between the first and second fixation means or the first and second bone plates as well as the pivoting element for pivoting the second fixation means or the second bone plate, a rigid connection existing between the distraction element and the pivoting element and further including a first retaining element fixed to the fixation element and a second retaining element fixed to the first fixation means or the first bone plate, and wherein the pivoting element has a threaded spindle and the first retaining element, which is fixed to the fixation element, has a through-bore with a thread for receiving the threaded spindle, and wherein the threaded spindle is received such that it cannot be displaced in the direction of the spindle axis in the second retaining element, which is fixed to the first fixation means or the first bone plate.

10. The distractor according to claim 4, wherein the hinge is formed such that the second fixation means or the second bone plate is pivotable in relation to the first fixation means or to the first bone plate by an absolute angle of up to 30°.

11. The distractor according claim 1 formed from titanium or some other biocompatible material.
